(19) United States
(12) Patent Application Publication
(10) Pub. No.: US 2024/0150727 A1
(43) Pub. Date: May 9, 2024

(54) SPECIES OF TOBAMOVIRUS

(71) Applicant: NUNHEMS B.V., Nunhem (NL)

(72) Inventors: Bart Peter Johan GERAATS, Nunhem (NL); Marco Antonio MAMMELLA, Sant Agata Bolognese (IT); Massimo TURINA, Torino (IT); Marina CIUFFO, Moncalieri (IT)

(21) Appl. No.: 18/342,484

(22) Filed: Jun. 27, 2023

Related U.S. Application Data
(63) Continuation of application No. 17/409,283, filed on Aug. 23, 2021, now abandoned.
Application No. 17/409,283 is a division of application No. 15/745,340, filed on Jan. 16, 2018, now Pat. No. 11,124,848.

(30) Foreign Application Priority Data
Jul. 17, 2015 (EP) 15177316.5

(51) Int. Cl. C12N 7/00; C07K 14/005; C07K 16/10; C12Q 1/70; G01N 33/50

(52) U.S. Cl. CPC C12N 7/00; C07K 14/005; C07K 16/10; C12Q 1/701; G01N 33/5097; C12N 2770/00021; C12N 2770/00022; C12N 2770/00031

(57) ABSTRACT

The present invention provides a new species of tobamovirus and its use to identify plants comprising resistance against the virus.

### CROSS REFERENCE TO RELATED APPLICATIONS

This application is a continuation of U.S. patent application Ser. No. 17/409,283, filed Aug. 23, 2021, which is a division of U.S. patent application Ser. No. 15/745,340 filed Jan. 16, 2018, which is a 35 U.S.C. 371 National Phase of PCT Application No. PCT/EP2016/066643 filed Jul. 13, 2016, which claims benefit to EP Application No. 15177316.5 filed Jul. 17, 2015, the disclosure of each of which is hereby incorporated by reference in their entireties.

### REFERENCE TO AN ELECTRONIC SEQUENCE LISTING

The contents of the electronic sequence listing (039621-01056 Sequence-Listing.xml; Size: 34,167 bytes; and Date of Creation: May 16, 2023) is herein incorporated by reference in its entirety.

### FIELD OF THE INVENTION

The invention relates to a new species of the genus tobamovirus (family Virgaviridae), which infects Solanaceae plants. The invention also relates to the use of the virus species for identifying and/or generating resistant Solanaceae plants, such as tomato, tobacco, pepper and eggplant. The new tobamovirus species is able to multiply and spread on tomato plants which carry Tomato Mosaic Virus (ToMV) resistance genes Tm1, Tm2 and Tm2², i.e. these resistance genes are ineffective against the new virus and plants develop various symptoms when infected, such as (mild) mosaic on leaves, bronzing of leaves, blistering and leaf distortions. Provided is a new virus species, called herein Tomato Mosaic Severe Virus (ToMSV or TMSV), methods for diagnosing the presence of the new tobamovirus species in plants and/or plant parts of Solanaceae species (especially tomato, tobacco, pepper and eggplant) and methods for using isolates of the new virus, such as isolate VE484 (deposited on Jan. 19, 2015 under accession number DSM 29970), to screen plants and/or plant parts for resistance against the virus.

### BACKGROUND OF THE INVENTION

Tobamoviruses in tomato were historically classified together as strains of Tobacco Mosaic Virus (TMV). However, nowadays these tobamoviruses are classified as different virus species based on sequence dissimilarity. Species of tobamoviruses include Pepper Mild Mottle Virus (PMMV), Tobacco Mosaic Virus (TMV), Tomato Mosaic Virus (ToMV), and many others (see e.g. ICTVdB Index of Viruses).

Plant viruses can be devastating to production of fruits and vegetables. Although many commercial varieties carry virus resistance genes, such resistance genes can become ineffective, as resistance breaking strains of a species may evolve or as new virus species may evolve. In tomato, three dominant ToMV resistance genes have been used for decades to control ToMV, namely Tm1 (introgression from S. habrochaites; conferring resistance to ToMV strains 0 and 2), Tm2 and Tm2² (both introgressions from S. peruvianum, conferring resistance against ToMV strains 0 and 1, and 0, 1 and 2, respectively). The Tm1 resistance gene encodes a protein that binds ToMV replication proteins and inhibits the RNA-dependent RNA replication of ToMV. The replication proteins of resistance-breaking mutants of ToMV do not bind Tm1, indicating that the binding is important for inhibition (see Ishibashi and Ishikawa, J Virol. July 2013; 87(14): 7933-7939). The Tm2² resistance gene encodes a protein that recognizes the ToMV movement protein, especially the carboxyterminus of the movement protein, and Tm2² resistance breaking strains with amino acid changes in the carboxyterminus of the movement protein are not recognized anymore and can thereby overcome resistance (see Weber and Pfitzner, 1998, MPMI Vol 11, pp 498-503). There is a constant evolutionary race between resistance genes and virus evolution. It is therefore important to identify new viruses quickly, in order to have tools to identify resistance genes which are effective against such new viruses.

It is an object of the invention to identify a new species of tobamovirus, referred herein as Tomato Mosaic Severe Virus (ToMSV or TMSV), which can infect tomato plants carrying any of these three resistance genes or combinations thereof. It is also an object of the invention to provide methods for diagnosing this new virus species, as well as methods of using virulent isolates of the species to screen germplasm for new resistance sources.

### BRIEF DESCRIPTION OF THE FIGURES

FIG. 1 shows the genome sequence of strain VE484 (SEQ ID NO: 1).

### DETAILED DESCRIPTION OF THE INVENTION

As used herein, the term “to comprise” and its conjugations is used in its non-limiting sense to mean that items following the word are included, but items not specifically mentioned are not excluded. Nevertheless, the skilled person will understand that the term “comprise” also encompasses the term “consists”. In addition, reference to an element by the recitation of “a” or “an” does not exclude the possibility that more than one of the element is present, unless the context clearly requires that there be one and only one of the elements. Thus, “a” or “an” usually means “at least one” or “one or more”.

The term “cultivar” (or “cultivated” plant) is used herein to denote a plant having a biological status other than a “wild” status, which “wild” status indicates the original non-cultivated, non-domesticated, or natural state of a plant or accession, and the term cultivated does not include such wild, or weedy plants. The term cultivar does include material with good agronomic characteristics, such as breeding material, research material, breeding lines, elite breeding lines, synthetic population, hybrid, founder stock/base population, inbred lines, cultivars (open pollinated of hybrid cultivar), segregating population, mutant/genetic stock, and advanced/improved cultivar. In one embodiment the term cultivar also includes landraces, i.e. pepper plants (or populations) selected and cultivated locally by humans over many years and adapted to a specific geographic environment and sharing a common gene pool. Cultivars have good agronomic properties compared to wild accessions such as high yielding, bigger fruit size, higher fertility, higher uniformity of plants and/or fruits, etc.

As used herein, the term “plant” includes the whole plant or any parts or derivatives thereof, such as plant organs (e.g., harvested or non-harvested fruits, leaves, seed, flowers, etc.), plant cells, plant protoplasts, plant cell or tissue cultures from which whole plants can be regenerated, plant calli, plant cell clumps, and plant cells that are intact in plants, or parts of plants, such as embryos, pollen, ovules, ovaries, fruits (e.g., harvested tissues or organs, such as harvested pepper fruits or parts thereof), flowers, leaves, seeds, clonally propagated plants, roots, root-stocks, stems, root tips and the like. Also any developmental stage is included, such as seedlings, immature and mature, etc.

“Plant variety” is a group of plants within the same botanical taxon of the lowest grade known, which (irrespective of whether the conditions for the recognition of plant breeder's rights are fulfilled or not) can be defined on the basis of the expression of characteristics that result from a certain genotype or a combination of genotypes, can be distinguished from any other group of plants by the expression of at least one of those characteristics, and can be regarded as an entity, because it can be multiplied without any change. Therefore, the term “plant variety” cannot be used to denote a group of plants, even if they are of the same kind, if they are all characterized by the presence of one locus or gene (or a series of phenotypical characteristics due to this single locus or gene), but which can otherwise differ from one another enormously as regards the other loci or genes.

Solanaceae refers to a family of plants, which include genera (especially the genus Solanum and the genus Capsicum) that comprise fruit and vegetable species which are cultivated and bred by humans, such as e.g. Solanum lycopersicum (tomato), Capsicum annuum (peppers), Solanum melongena (aubergine) and Solanum muricatum (pepino).

“Tomato plants” or “cultivated tomato plants” are plants of the Solanum lycopersicum, i.e. varieties, breeding lines or cultivars of the species Solanum lycopersicum, cultivated by humans and having good agronomic characteristics; preferably such plants are not “wild plants”, i.e. plants which generally have much poorer yields and poorer agronomic characteristics than cultivated plants and e.g. grow naturally in wild populations. “Wild plants” include for example wild accessions or wild relatives of a species. In one aspect of the invention so-called heirloom tomato varieties or cultivars, i.e. open pollinated varieties or cultivars commonly grown during earlier periods in human history and often adapted to specific geographic regions, are in one aspect of the invention encompassed herein as cultivated tomato plants. In one embodiment the term cultivar also includes landraces, i.e. plants (or populations) selected and cultivated locally by humans over many years and adapted to a specific geographic environment and sharing a common gene pool.

The term “cultivar” (or “cultivated” plant) is used herein to denote a plant having a biological status other than a “wild” status, which “wild” status indicates the original non-cultivated or natural state of a plant or accession, and the term cultivated does not include such wild, or weedy plants. The term cultivar does include material with good agronomic characteristics, such as breeding material, research material, breeding lines, elite breeding lines, synthetic population, hybrid, founder stock/base population, inbred lines, cultivars (open pollinated of hybrid cultivar), segregating population, mutant/genetic stock, and advanced/improved cultivar.

Wild relatives of tomato include S. arcanum, S. chmielewskii, S. neorickii (=L. parviflorum), S. cheesmaniae, S. galapagense, S. pimpinellifolium, S. chilense, S. corneliomulleri, S. habrochaites (=L. hirsutum), S. huaylasense, S. sisymbriifolium, S. peruvianum, S. hirsutum, S. pennellii, S. lycopersicoides, S. sitiens or S. ochranthum.

As used herein, “pepper plant” or “pepper” is a plant of genus Capsicum or parts thereof (e.g. fruits). Pepper includes all kinds of peppers, such as hot/pungent peppers and non-pungent peppers (sweet pepper). The term encompasses wild accessions and domesticated peppers.

“Domesticated pepper” refers to the species Capsicum annuum L., Capsicum chinense Jacq., Capsicum frutescens L., Capsicum baccatum L., and Capsicum pubescens Ruiz & Pay. The term “cultivated pepper” refers to breeding lines and varieties of domesticated pepper, which is cultivated by humans in the field or in protected environments (e.g. greenhouse or tunnels) for fruit production. Cultivars have good agronomic properties compared to wild accessions such as high yielding, bigger fruit size, higher fertility, higher uniformity of plants and/or fruits, etc. Examples of cultivars include cultivated varieties that belong to the species Capsicum annuum, Capsicum chinense, Capsicum frutescens, Capsicum baccatum and Capsicum pubescens.

A “plant genotype” refers to plants that are genotypically closely related, such as plants of an accession of a seed-bank (e.g. in the GRIN collection of the USA; http://www.ars-grin.gov/npgs/acc/acc_queries.html or the CGN (Centre for Genetic Resources) collection of Wageningen University and Research Centre in the Netherlands) and progeny thereof obtained by selfing, or plants of a plant line, or a plants of a variety.

A “plant line” or “breeding line” refers to a plant and its progeny. As used herein, the term “inbred line” refers to a plant line which has been repeatedly selfed.

“F₁, F₂, etc.” refers to the consecutive related generations following a cross between two parent plants or parent lines. The plants grown from the seeds produced by crossing two plants or lines is called the F₁ generation. Selfing the F₁ plants results in the F₂ generation, etc. “F₁ hybrid” plant (or F₁ hybrid seed) is the generation obtained from crossing two inbred parent lines.

“Hybrid” or “hybrid plant” is a plant produced by the intercrossing (cross-fertilization) of at least two different plants or plants of different parent lines. It is understood that the seeds of such a cross (hybrid seeds) are encompassed herein, as well as the hybrid plants grown from those seeds and plant parts derived from those grown plants.

The term “trait” refers to a heritable characteristic, such as ToMSV resistance, which is transferrable, e.g., by crossing and selection from one plant to another.

“ToMSV”, “ToMSV strains” or “ToMSV isolates” or “ToMSV pathotypes” refers to strains of Tomato Mosaic Severe Virus which can be determined serologically (using antibodies), by microscopy, by sequence comparison, and/or by disease assays, all as described herein.

“VE484” refers to a virulent strain of ToMSV, a representative sample of which has been deposited at the DSZM under Accession number DSM29970.

“Tm1 gene” or “Tm1 resistance gene” refers to the dominant resistance gene as known in the art, conferring resistance to ToMV strains 0 and 2, e.g., obtainable from commercial tomato varieties Mobaci.

“Tm2 gene” or “Tm2 resistance gene” refers to the dominant resistance gene as known in the art, conferring resistance to ToMV strains 0 and 2, e.g., obtainable from commercial tomato varieties Moperon.

“Tm2² gene” or “Tm2² resistance gene” refers to the dominant resistance gene as known in the art, conferring resistance to ToMV strains 0, 1 and 2, e.g., obtainable from commercial tomato varieties Momor.

A plant (e.g. a Solanaceae, e.g. a wild relative of tomato or a cultivated tomato, a wild or cultivated Capsicum species, a aubergine or a pepino) having or comprising “ToMSV-resistance” or being “ToMSV-resistant” refers to a plant which does not develop systemic symptoms and in which the virus does, or does not, spread systemically (i.e. to non-inoculated and/or non-infected parts of the plant, such as upper leaves) after inoculation or infection with one or more infectious strains of ToMSV, such as strain V484. If the virus does not develop systemic symptoms and also cannot spread systemically, the resistance cane be called “complete resistance”, while if the virus does not develop systemic symptoms but the virus can spread systemically, this resistance can also be called “tolerance” herein. Resistance (including complete resistance and tolerance) can be tested using various methods, one example is using an artificial mechanical inoculation assay, whereby, for example, the one or two young leaves or cotyledons of a plant are mechanically inoculated with an infectious strain (e.g. Ve484), and the non-inoculated plant parts (such as upper leaves) are evaluated one or more days post inoculation for systemic symptoms (such as mosaic, leaf distortion, blistering and/or bronzing) and/or for presence of the virus in non-inoculated plant parts (using e.g. ELISA, electron microscopy, etc.).

“Systemic symptoms” are symptoms that can be seen on other tissues or plant parts (e.g. other leaves) than the inoculated/infected tissue or plant part (e.g. true leaf or cotyledon or stem or hypocotyl), e.g. on upper leaves to which the virus has spread from the inoculated/infected leaf or cotyledon or stem or hypocotyl.

“Systemic spread” refers to the virus having spread from the inoculated/infected tissue or plant part (e.g. leaf or cotyledon or stem or hypocotyl) to non-inoculated/non-infected tissues or plant parts, e.g. to upper leaves.

The term “allele(s)” means any of one or more alternative forms of a gene at a particular locus, all of which alleles relate to one trait or characteristic at a specific locus. In a diploid cell of an organism, alleles of a given gene are located at a specific location, or locus (loci plural) on a chromosome. One allele is present on each chromosome of the pair of homologous chromosomes. A diploid plant species, such as pepper and tomato, may comprise a large number of different alleles at a particular locus. These may be identical alleles of the gene (homozygous) or two different alleles (heterozygous).

The term “protein” refers to a polypeptide having a mode of action, size, three-dimensional structure or origin. A “fragment” or “portion” of a protein may thus still be referred to as a “protein”. An “isolated protein” is used to refer to a protein which is no longer in its natural environment.

“Sequence identity” and “sequence similarity” can be determined by alignment of two peptide or two nucleotide sequences using global or local alignment algorithms. Sequences may then be referred to as “substantially identical” or “essentially similar” when they (when optimally aligned by for example the programs GAP or BESTFIT or the Emboss program “Needle” (using default parameters, see below) share at least a certain minimal percentage of sequence identity (as defined further below). These programs use the Needleman and Wunsch global alignment algorithm to align two sequences over their entire length, maximizing the number of matches and minimises the number of gaps. Generally, the default parameters are used, with a gap creation penalty=10 and gap extension penalty=0.5 (both for nucleotide and protein alignments). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may for example be determined using computer programs, such as the GCG Wisconsin Package, Version 10.3, available from Accelrys Inc., 9685 Scranton Road, San Diego, CA 92121-3752 USA or EMBOSS (http://www.ebi.ac.uk/Tools/web services/services/emboss). Alternatively percent similarity or identity may be determined by searching against databases such as FASTA, BLAST, etc., but hits should be retrieved and aligned pairwise to compare sequence identity.

“Average” refers herein to the arithmetic mean.

“Stringent hybridisation conditions” can be used to identify nucleotide sequences (in method known as nucleic acid hybridization methods), which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) Molecular Cloning: A Laboratory Manual, Second Edition, Cold Spring Harbor Laboratory Press, and Sambrook and Russell (2001) Molecular Cloning: A Laboratory Manual, Third Edition, Cold Spring Harbor Laboratory Press, NY; and in Volumes 1 and 2 of Ausubel et al. (1994) Current Protocols in Molecular Biology, Current Protocols, USA.

### DETAILED DESCRIPTION OF THE INVENTION

The present invention provides a new tobamovirus, referred herein to as Tomato Mosaic Severe Virus (ToMSV or TMSV), which was identified in a leaf sample from a tomato production field and was identified to be a tobamovirus by ELISA, electron microscopy and bioassays, and was found to be a new species of tobamovirus by sequencing of the viral genome. A strain of the virus was isolated and is referred to as strain VE484, which was deposited at the DSMZ. The strain VE484 is able to infect cultivated tomato plants comprising the widely used resistance genes Tm1, Tm2 and Tm2². It is also able to infect other Solanaceae, especially members of the genus Capsicum, such as cultivated pepper, and likely other members of the genus Solanum, besides Solanum lycopersicum (tomato), such as wild relatives of tomato, Solanum melongena (aubergine) and Solanum muricatum (pepino).

As in other tobamoviruses, the genome sequence (SEQ ID NO: 1 and SEQ ID NO: 2) of this positive sense single stranded RNA virus encodes four ORFs (Open Reading Frames). The entire genome is 6402 bases long and has only 82% sequence identity to the most similar virus, Genbank accession FR878069.1 (Tobacco Mosaic Virus Strain Ohio V, complete genome, genomic RNA). According to this low percentage (less than 90%) of genome sequence identity a new species designation within the genus tobamoviruses is therefore proposed, which is in accordance with the species demarcation criteria proposed in the book “Virus Taxonomy: 9^{th} report of the International Committee on Virus Taxonomy”, ISBN 978-0-12-384684-6, p 1155. Herein viruses with nucleotide genome sequences with less than 90% sequence identity are proposed to be classified as a new genus.

Also the proteins encoded by the four ORFs are unique in public sequence databases. The percentage sequence identity to the most similar Genbank accessions is shown below, using Emboss-needle (pairwise alignments, default parameters):

TABLE 1
ORF1 (SEQ ID NO: 3), protein p126

| | ORF1 (SEQ | | |
|---|---|---|---|
| | ID NO: 3) | NP_078447.1 | AHW98774.1 |
| ORF1 (SEQ ID NO: 3) | 100% | | |
| NP_078447.1 | 92.7% | 100% | |
| AHW98774.1 | 92.7 | 99.7% | 100% |

The protein p126 (SEQ ID NO: 3) is 1116 amino acids in length, has a molecular weight of 126 kDa and contains a methyltransferase and a helicase domain.

TABLE 2
ORF2 (SEQ ID NO: 4), protein p183

| | ORF2 (SEQ | | |
|---|---|---|---|
| | ID NO: 4) | CCC33060.1 | ABN79257.1 |
| ORF2 (SEQ ID NO: 4) | 100% | | |
| CCC33060.1 | 93.0% | 100% | |
| ABN79257.1 | 92.9% | 98.8% | 100% |

The protein p183 (SEQ ID NO: 4) is 1609 amino acids in length and has a molecular weight of 183 kDa and contains a methyltransferase and a helicase domain (as does p126) and additionally a polymerase domain (RdRP). Protein p183 results from suppression of the p126 stop codon.

TABLE 3
ORF3 (SEQ ID NO: 5), Movement Protein

| | ORF3 (SEQ | | |
|---|---|---|---|
| | ID NO: 5) | AAY44881.1 | CCC33061.1 |
| ORF3 (SEQ ID NO: 5) | 100% | | |
| AAY44881.1 | 79.4% | 100% | |
| CCC33061.1 | 79.4% | 96.3% | 100% |

The protein of SEQ ID NO: 5 (ORF3) is 266 amino acids in length, has a molecular weight of 29.7 kDa, and is the viral movement protein (MP).

TABLE 4
ORF4 (SEQ ID NO: 6), Coat Protein

| | ORF4 (SEQ | | |
|---|---|---|---|
| | ID NO: 6) | AIW42686.1 | ABN13962.1 |
| ORF4 (SEQ ID NO: 6) | 100% | | |
| ABN13962.1 | 81.8 | 100% | |
| AIW42686.1 | 80.7 | 96.9 | 100% |

The protein of SEQ ID NO: 6 (ORF4) is 176 amino acids in length, has a molecular weight of 19.6 kDa, and is the viral Coat Protein (CP).

The virus is not insect-vector transmitted, but is mechanical transmitted. It might also be transmitted via seeds (i.e. seeds produced on infected plants) as the virus was found to be seed born.

Electron microscopy of infected leaves showed that virus particles are rod-shaped, with about 300 nanometer (nm) length and 18 nm diameter. The virus can be propagated on Solanaceae, e.g. S. lycopersicum or Nicotiana benthamiana, using mechanical inoculation of leaves.

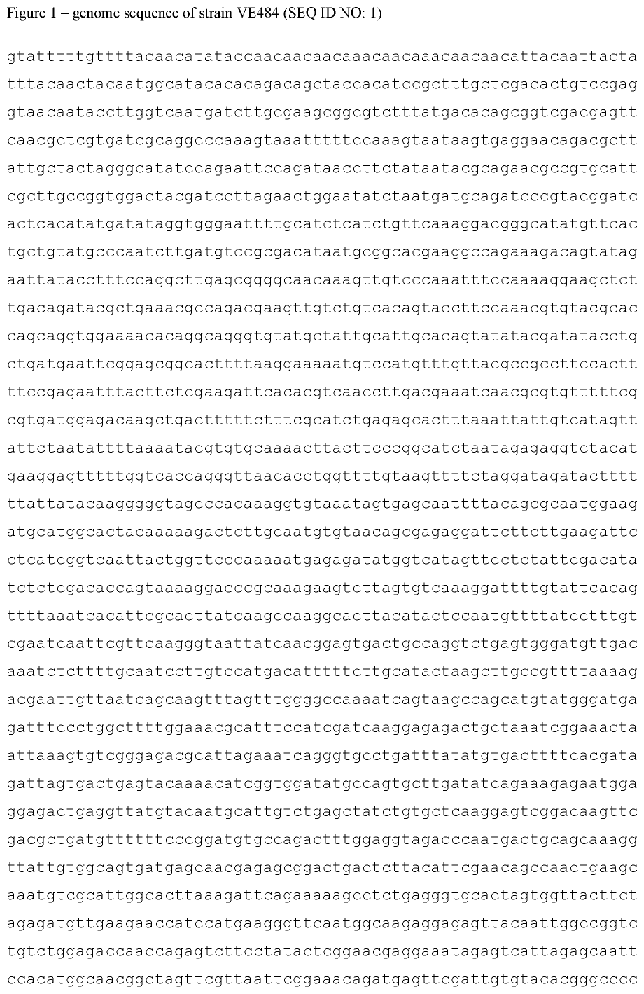

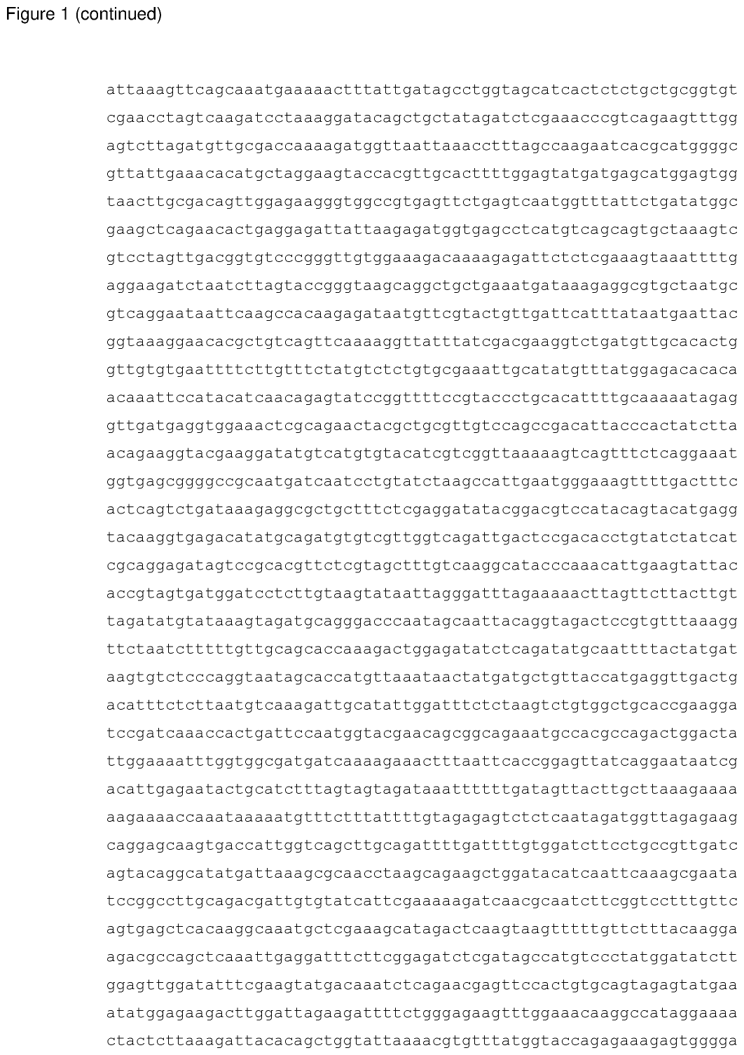

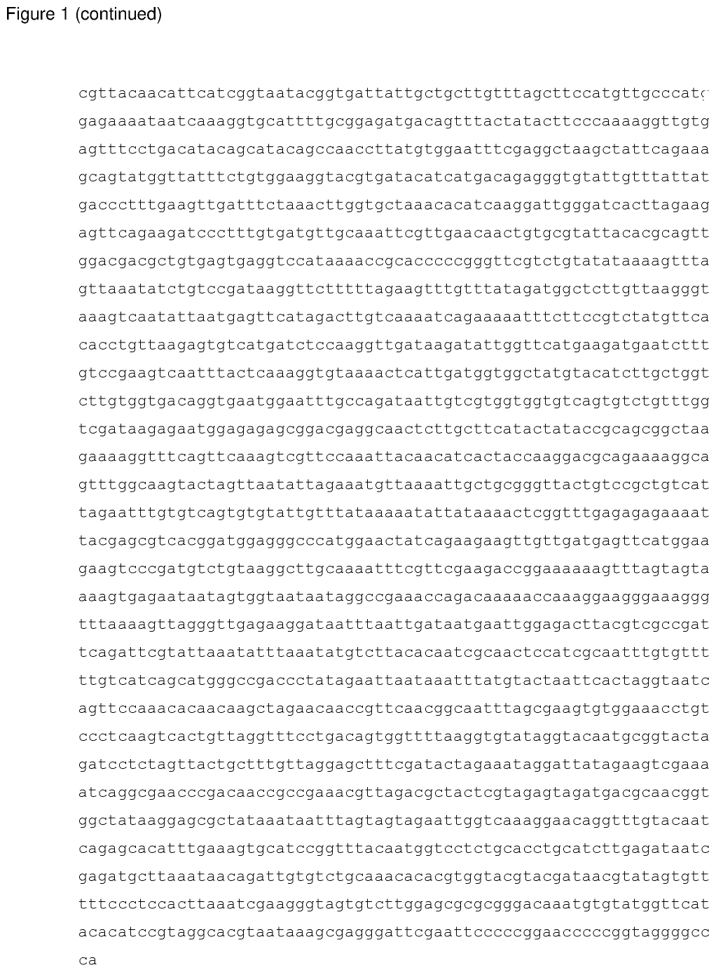

In one aspect the invention provides a new species of tobamovirus whose genome sequence comprises substantial sequence identity to SEQ ID NO: 1, i.e. at least 83% sequence identity to SEQ ID NO:1, preferably at least 84%, 85%, 86%, 87%, 88%, 89%, 90%, 91%, 92%, 93%, 94%, 95%, 96%, 97%, 98% or even 99% or 100%. In one aspect the invention provides a new species of tobamovirus whose nucleic acid genome sequence comprises substantial sequence identity to SEQ ID NO: 1, wherein substantial sequence identity means at least 90%, preferably at least 91%, 92%, 93% or more (e.g. at least 94%, 95%, 96%, 97%, 98% or even 99% or 100%) sequence identity to SEQ ID NO: 1 (also shown in FIG. 1). The sequence identity is determined by using a pairwise alignment of the entire genome sequences, using e.g. the Emboss program ‘Needle’ (using default parameters). Apart from strain VE484 (a representative sample of which has been deposited), other strains of this new species exist or will develop. The skilled person can easily isolate and identify such other strains based on the instant invention, such as one or more criteria selected from sequence identity, bioassays, symptoms, antibody based assays, etc.

In one aspect the tobamovirus is provided whereby it is not present in a living plant (i.e. the virus is isolated from a whole living plant such as a plant found in the field), but is provided in e.g. a severed plant part such as fresh leaf tissue, freeze dried plant tissue, or in extracted plant sap or in a solution such as a buffer. It is also provided in a container comprising the virus.

The virus is infectious on Solanaceae species, especially on species of the genus Solanum and Capsicum. In one aspect the virus causes systemic symptoms on species of the genus Solanum, such as at least on Solanum lycopersicum, especially cultivated S. lycopersicum lacking Tm resistance genes or comprising one or more Tm resistance genes selected from Tm1, Tm2 and Tm2². The virus for example causes systemic symptoms on tomato cultivars having the following genotypes: Tm1/Tm1 (homozygous for Tm1); Tm1Tm2/Tm1Tm2 (homozygous for Tm1 and Tm2); Tm1/Tm2²/Tm1/Tm2² (homozygous for Tm1 and Tm2²); Tm2/Tm2 (homozygous for Tm1); Tm2²/Tm2² (homozygous for Tm2²).

In a further aspect the virus causes systemic symptoms on plants of the genus Capsicum, especially on cultivated pepper of the species C. annuum.

Systemic symptoms are symptoms in other parts of plant than where the virus entered or was inoculated. This means the virus is able to spread from an infection site (e.g. a leaf) to other parts of the plant, e.g. other leaves, such as upper leaves (i.e. the virus spreads systemically). The systemic symptoms vary, and include one or more of: mosaic, leaf distortion, leaf blistering and/or leaf bronzing. In some instances plants also remain asymptomatic, even though the virus has spread systemically. Therefore, in one aspect the ability of the virus to cause systemic symptoms means that in at least 40%, 50%, 60%, preferably in at least 70%, 80%, 85%, 90% or more of plants of the same genotype which are infected by the virus (e.g. in the field or via seed transmission) or inoculated (e.g. mechanically inoculated) one or more systemic symptoms develop. Such a plant genotype is thus susceptible to the virus, even though not all plants show systemic symptoms.

In both symptomatic and in asymptomatic plants the systemic spread of the virus can be determined by various methods, which detect (and optionally quantify) the presence of the virus in non-infected or non-inoculated parts of the plant, such as in upper leaves. The virus can be detected by various methods or combinations thereof, including microscopy (e.g. Electron Microscopy), antibody based tests such as ELISA or Lateral Flow Device tests, RT-PCT (reverse transcriptase PCR), sequencing, nucleic acid hybridization methods (using e.g. stringent hybridization conditions), bioassays, e.g. inoculation of indicator plants such as Nicotiana tabacum var. Xanthi (comprising the N gene in homozygous form) and Nicotiana glutinosa, etc.

In a further aspect a container comprising the new species of tobamovirus is provided. Preferably the container comprises one strain of the virus, preferably an infectious strain. A container may be any container, such as a tube, a vial, a well, a bottle, bag, etc. The virus may be present in the container in various forms, such as in severed plant tissue (e.g. a fresh, dried or lyophilized tissue, such as leaf or leaf part, stem or stem part, seed or seed part, etc.). The virus may also be present outside of plant tissue, e.g. in a liquid, such as extracted plant sap, or in a solution not comprising plant cells and not comprising plant sap, such as water, or a buffer solution.

In one aspect a sterilized solution comprising the virus according to the invention is provided. In one aspect the virus is a single infectious strain, such as VE484 or any other ToMSV strain. In one aspect the solution is a buffer solution.

Also provided is a method of using the virus according to the invention for identifying plants of the genus Solanum or of the genus Capsicum which comprise resistance against the virus, either complete resistance or tolerance. The new virus can be used to screen different plant genotypes of the genus Solanum and/or Capsicum for plant genotypes which are resistant (completely resistant or tolerant). In one aspect this means that the virus does not cause systemic symptoms and does not spread systemically (complete resistance) on a plant genotype. The virus may optionally cause local lesions on inoculated plant parts (e.g. leaves) of the genotype. In another aspect the virus does not cause systemic symptoms but does spread systemically (tolerance) on a plant genotype.

The methods can be used to identify a cultivated plant (e.g. cultivated tomato, S. lycopersicum, or cultivated pepper, Capsicum annuum) or preferably a wild plant of the genus Solanum or Capsicum comprising ToMSV resistance. Thus, in one aspect the method is used to identify a ToMSV resistant plant (e.g. a VE484 resistant plant) wherein the plant is selected from the species S. lycopersicum, S. arcanum, S. chmielewskii, S. neorickii (=L. parviflorum), S. cheesmaniae, S. galapagense, S. pimpinellifolium, S. chilense, S. corneliomulleri, S. habrochaites (=L. hirsutum), S. huaylasense, S. sisymbriifolium, S. peruvianum, S. hirsutum, S. pennellii, S. lycopersicoides, S. sitiens or S. ochranthum.

In another aspect the method is used to identify a ToMSV resistant plant (e.g. a VE484 resistant plant) wherein the plant is selected from the species Capsicum annuum L., Capsicum chinense Jacq., Capsicum frutescens L., Capsicum baccatum L., and Capsicum pubescens.

In yet another aspect the method is used to identify a ToMSV resistant plant (e.g. a VE484 resistant plant) wherein the plant is selected from the species Solanum melongena or

Solanum muricatum.

The method comprises the steps of:
a) providing one or more plants;
b) providing inoculum comprising the virus according to the invention;
c) inoculating one or more plant parts of the plants of a) with the inoculum of b);
d) incubating the inoculated plants.

Thus, as mentioned, the one or more plants of step a) are preferably one or more plants of the genus Solanum or Capsicum, such as one or more plants of the species mentioned above. When reference to “one or more plants” is made it is understood that preferably several plants of one or more plant genotypes is referred to. So for example if one or more genotypes (or accessions) are tested, such as one or more wild accessions of relatives of tomato, preferably several plants of each genotype are provided, e.g. at least 2, 3, 4, 5, 6, 7, 8, 9, 10, 11, 12, 13, 14, or more plants of each genotype. Similarly if one or more breeding lines or cultivars are to be tested, several plants of each breeding line or cultivar are provided. Preferably also a ToMSV susceptible plant genotype with known symptomatology is included, such as tomato variety Mobaci, Moperou, Momor, Mocimor, Philippos or others.

An inoculum of the virus can be made using known methods. E.g. symptomatic leaves or other infected plant tissue can be collected and ground in the presence of a buffer or infectious plant sap can be used. The virus as deposited can also be used to make infectious inoculum. It is noted that the skilled person does not need to use the virus strain as deposited (VE484, DSM29970), but can identify a strain of the new tobamovirus in the field, optionally verifying the identity by sequencing the virus strain, and can use the strain to make infectious inoculum.

Step c) is preferably carried out by mechanical inoculation, i.e. the plant surface is mechanically damaged slightly to allow the virus to enter. So, for example one or more leaves or cotyledons of each plant may be dusted with e.g. carborundum powder before the inoculum is added to the leaf surface or cotyledon. Alternatively, other plant parts may be inoculated, such as a part of the stem, the hypocotyl, the petiole or the root. It is clear that there are different ways to slightly damage the plant surface. Thus, in one aspect the plant part of c) is a leaf, a cotyledon, a stem, a hypocotyl, a root or a petiole.

In step d) the plants are then incubated at a temperature, light and relative humidity which allows the plants to grow further (depending on the species). The plants may then be inspected regularly for systemic symptoms and/or for local lesions, e.g. after at least 1, 2, 3, 4, 5, 6, 7, 8, 9, 10, 11, 12, 13, 14, 15, 16, 17, 18 and/or more days post-inoculation.

Thus, in one aspect the method further comprises step e): assessing symptoms on the plants or plant parts, especially systemic symptoms and/or local lesions (on the inoculated plant part). “Local lesions” or “local necrotic lesions” are lesions which form as a defence reaction of the plant and which prevent the spread of the virus to non-inoculated parts. Thus systemic virus symptoms such as one or more of mosaic, leaf distortion, blistering and/or bronzing may be assessed, especially on non-inoculated parts of the plant, such as upper leaves. Symptom assessment can be made visually. Optionally the method further comprises step f) determining (or assessing) the presence of the virus particles in non-inoculated plant parts, such as upper true leaves. This can be done on one or more or all of the inoculated plants. In one aspect it is carried out on one or more plants which show no systemic symptoms.

In one aspect step e) above is omitted, i.e. symptoms are not assessed but the presence of virus particles in (one or more) non-inoculated plant parts is assessed one or more of the inoculated plants.

Following step e) or f) one can identify (make a selection of) a resistant or of a tolerant plant, or alternatively discard all susceptible plants. The identification can be made in various ways:

In one aspect the method further comprises identifying a plant (or a number of plants) which has no systemic symptoms and in which the virus particles are not present in non-inoculated plant parts. Such plants can be considered resistant against the virus.

In another aspect the method further comprises identifying a plant (or a number of plants) which has local lesions on the inoculated plant part and/or in which the virus particles are not present in non-inoculated plant parts. Such plants can be considered resistant against the virus.

In yet another aspect the method further comprising identifying a plant which has no systemic symptoms and in which the virus particles are present in non-inoculated parts of the plant. Such a plant can be considered tolerant against the virus, i.e. even though the virus is capable of spreading systemically, it does not cause systemic symptoms.

In still another aspect the method further comprising identifying a plant which has significantly reduced systemic symptoms compared to susceptible control plants and in which the virus particles are present in non-inoculated parts of the plant. Such a plant can be considered as being partially resistant against the virus. Significantly reduced systemic symptoms may for example be that the percentage of plant of the genotype showing one or more systemic symptoms is statistically significantly lower than the percentage of susceptible control plants showing one or more systemic symptoms. So, for example if 100% of plants of the susceptible control genotype show one or more systemic symptoms, in a partially resistant genotype significantly less plants show one or more systemic symptoms, such as less than 90%, 80%, 70%, 60%, 50% or even less than 40% of the inoculated plants show one or more systemic symptoms.

The presence of virus particles in non-inoculated plant parts can be determined in various ways and can optionally be quantified. So, for example an ELISA test as described in the Examples can be carried out on non-inoculated parts, e.g. upper leaves of the plants in order to determine whether the virus is present and has thus spread systemically. Different methods can be used to detect the virus, such as microscopy (e.g. Electron Microscopy), antibody based assays (e.g. ELISA or Lateral Flow Device Tests), or tests which detect virus RNA (or cDNA), such as Polymerase Chain Reaction (PCR) based methods, nucleic acid sequence, nucleic acid hybridization methods, or bioassays e.g. inoculation of indicator plants such as Nicotiana tabacum var. Xanthi (comprising the N gene in homozygous form) and Nicotiana glutinosa, etc.

Optionally plants which do not contain the virus in non-inoculated plant parts may be selected and/or plants do contain the virus in non-inoculated plant parts may be discarded. Thus in one aspect the method comprises determining the presence of virus particles in non-inoculated plant parts.

The identified plant (or plant genotype) may also be re-tested in the above method(s), i.e. one or more identified plants may be inoculated again with the virus and incubated again, to confirm their resistance or partial resistance against the ToMSV strain in a second assay or third assay. Also different ToMSV strains may be tested. As in the initial screen it may have been that only a few plants of a genotype are tested (e.g. 1, 2, 3, 4 plants) and as only 1 or a few plants of a genotype may be identified and selected in the initial screen, it is preferred that in a second or third screen a larger number of plants of the identified genotype are re-tested, such as at least 10, 11, 12, 13, 14, 15, 16, 17, 18, 19, 20, 21, 22, 23, 34, 25, 26, 27, 28, 29, 30 or more.

When a larger number of plants of a given genotype are tested in any of the above methods, preferably at least 80%, 90%, 91%, 92%, 93%, 94%, 95%, 97%, 98%, 99% or most preferably 100% of plants of the same genotype are identified as being completely resistant. Likewise in one aspect preferably at least 80%, 90%, 91%, 92%, 93%, 94%, 95%, 97%, 98%, 99% or most preferably 100% of plants of the same genotype are identified as being tolerant.

The above methods can, therefore, be used to identify plants, especially wild relatives of tomato or wild accessions of Capsicum, or plants of aubergine or pepino, comprising resistance against one or more strains of ToMSV, e.g. against VE484. Plants obtained by the above method are encompassed herein. The resistance genes found in such plants can then be used to make cultivated tomato plants or cultivated pepper plants or cultivated aubergine or pepino comprising resistance against one or more strains of ToMSV, e.g. against at least against strain VE484. Such plants can be made by traditional breeding techniques.

In a further aspect the invention provides a method for assessing the presence of a virus according to the invention in a plant or plant part, said method comprising the steps of:
a) determining the presence of a nucleic acid molecule comprising at least 83% sequence identity to SEQ ID NO:1 in the plant or plant part; and/or
b) determining the presence of a protein, or of a nucleic acid molecule encoding a protein, comprising at least 93% sequence identity to SEQ ID NO: 3; and/or
c) determining the presence of a protein, or of a nucleic acid molecule encoding a protein, comprising at least 94% sequence identity to SEQ ID NO: 4; and/or
d) determining the presence of a protein, or of a nucleic acid molecule encoding a protein, comprising at least 80% sequence identity to SEQ ID NO: 5; and/or
e) determining the presence of a protein, or of a nucleic acid molecule encoding a protein, comprising at least 82% sequence identity to SEQ ID NO: 6.

As the virus is an RNA virus, the nucleic acid molecule is an RNA molecule. RNA molecules are generally detected by first reverse transcribing the RNA into DNA (complementary DNA or cDNA) and then detecting the cDNA. RNA and cDNA have the same nucleotide base sequence, except that Uracil (U) of RNA is replaced by Thymine (T). Therefore detection of the cDNA is equivalent to detection of the RNA molecule.

The presence of a nucleic acid molecule according to any of steps a), b), c), d) or e) can be easily determined by the skilled person using known methods. For example, the skilled person can design a pair of primers for RT-PCR and carry out an RT-PCR assay to amplify the nucleic acid molecule or part thereof. Also nucleic acid hybridization methods may be used (using e.g. stringent conditions).

Regarding step a) the primers can be designed to amplify any part of SEQ ID NO: 1 (the nucleic acid sequence of the genome of VE484) or of a nucleic acid molecule comprising at least 83% sequence identity to SEQ ID NO:1, e.g. at least 84%, 85%, 86%, 87%, 88%, 89%, 90%, 91%, 92%, 93%, 94%, 95%, 96% or 97%, 98% or 99% sequence identity to SEQ ID NO: 1. Optionally the primers of primer pair SEQ ID NO: 7 and 8 or SEQ ID NO: 9 and 10 can be used.

For detection of a nucleic acid sequence encoding a comprising at least 93% (e.g. at least 94%, 95%, 96%, 97%, 98%, 99% or 100%) sequence identity to SEQ ID NO: 3; and/or comprising at least 94% (e.g. at least 95%, 96%, 97%, 98%, 99% or 100%) sequence identity to SEQ ID NO: 4; and/or comprising at least 80% (e.g. at least 81%, 82%, 83%, 84%, 85%, 86%, 87%, 88%, 89% 90%, 91%, 92%, 93%, 94%, 95%, 96%, 97%, 98%, 99% or 100%) sequence identity to SEQ ID NO: 5; and/or comprising at least 82% (e.g. at least 83%, 84%, 85%, 86%, 87%, 88%, 89% 90%, 91%, 92%, 93%, 94%, 95%, 96%, 97%, 98%, 99% or 100%) sequence identity to SEQ ID NO: 6, various methods can be used, such as also the design of primer pairs (or degenerate primer pairs) which amplify such nucleotide sequences and detect the presence of an RNA molecule having that sequence in a sample. Again RT-PCR can for example be used. Also nucleic acid hybridization methods may be used (using e.g. stringent conditions).

The nucleotide sequences encoding the protein of SEQ ID NO: 3, 4, 5 and 6 are shown in SEQ ID NO: 1 and 2. The protein of SEQ ID NO: 3 is encoded by nucleotides 77 to 3424 of SEQ ID NO: 1. The protein of SEQ ID NO: 4 is encoded by nucleotides 77 to 3424 and by 3446 to 4921 of SEQ ID NO:1. The protein of SEQ ID NO: 5 is encoded by nucleotides 4911 to 5708 of SEQ ID NO: 1 and the protein of SEQ ID NO: 6 is encoded by nucleotides 5671 to 6198 of SEQ ID NO: 1.

Regarding the determination of the presence of a protein comprising at least 93% (e.g. at least 94%, 95%, 96%, 97%, 98%, 99% or 100%) sequence identity to SEQ ID NO: 3; and/or comprising at least 94% (e.g. at least 95%, 96%, 97%, 98%, 99% or 100%) sequence identity to SEQ ID NO: 4; and/or comprising at least 80% (e.g. at least 81%, 82%, 83%, 84%, 85%, 86%, 87%, 88%, 89% 90%, 91%, 92%, 93%, 94%, 95%, 96%, 97%, 98%, 99% or 100%) sequence identity to SEQ ID NO: 5; and/or comprising at least 82% (e.g. at least 83%, 84%, 85%, 86%, 87%, 88%, 89% 90%, 91%, 92%, 93%, 94%, 95%, 96%, 97%, 98%, 99% or 100%) sequence identity to SEQ ID NO: 6, various methods can be used. In one aspect preferably antibody based methods are used, such as ELISA or LFD. One can use any capture antibody which can bind one of the above proteins (to form an antibody-antigen complex). The proteins or protein parts may be used as antigens i.e. to raise and make antibodies which bind the proteins. The examples also show that the TMV antibody sold by Agdia, Inc. (Catalog numbers CAB57400, ECA 57400, PSA 57400 and SRA57400), can be used to detect the strain VE484.

In another aspect an isolated nucleic acid molecule comprising at least 83% (e.g. at least 84%, 85%, 86%, 87%, 88%, 89% 90%, 91%, 92%, 93%, 94%, 95%, 96%, 97%, 98%, 99% or 100%) sequence identity to SEQ ID NO:1, or a part thereof, is provided. The nucleic acid molecule may be a RNA or DNA molecule. A part thereof may be a fragment, such as any molecule comprising at least 15, 20, 30, 40, 50, 100, 200, 500, 1000, 1500, 2000, 2500, 3000, 3500, 4000, 4500, or 6000 consecutive nucleotides of such a nucleic acid molecule.

In still a different aspect an isolated nucleic acid molecule comprising at least 80% (e.g. at least 81%, 82%, 83%, 84%, 85%, 86%, 87%, 88%, 89% 90%, 91%, 92%, 93%, 94%, 95%, 96%, 97%, 98%, 99% or 100%) to nucleotides 77 to 3424 of SEQ ID NO: 1 is provided.

In still a different aspect an isolated nucleic acid molecule comprising at least 80% (e.g. at least 81%, 82%, 83%, 84%, 85%, 86%, 87%, 88%, 89% 90%, 91%, 92%, 93%, 94%, 95%, 96%, 97%, 98%, 99% or 100%) to nucleotides 3446 to 4921 of SEQ ID NO:1 is provided.

In still a different aspect an isolated nucleic acid molecule comprising at least 80% (e.g. at least 81%, 82%, 83%, 84%, 85%, 86%, 87%, 88%, 89% 90%, 91%, 92%, 93%, 94%, 95%, 96%, 97%, 98%, 99% or 100%) to nucleotides 4911 to 5708 of SEQ ID NO: 1 is provided.

In still a different aspect an isolated nucleic acid molecule comprising at least 80% (e.g. at least 81%, 82%, 83%, 84%, 85%, 86%, 87%, 88%, 89% 90%, 91%, 92%, 93%, 94%, 95%, 96%, 97%, 98%, 99% or 100%) to nucleotides 5671 to 6198 of SEQ ID NO: 1.

Also provided is a protein selected from the group consisting of: a protein comprising at least 93% (e.g. at least 94%, 95%, 96%, 97%, 98%, 99% or 100%) sequence identity to SEQ ID NO: 3, a protein comprising at least 94% (e.g. at least 95%, 96%, 97%, 98%, 99% or 100%) sequence identity to SEQ ID NO: 4, a protein, comprising at least 80% (e.g. at least 81%, 82%, 83%, 84%, 85%, 86%, 87%, 88%, 89% 90%, 91%, 92%, 93%, 94%, 95%, 96%, 97%, 98%, 99% or 100%) sequence identity to SEQ ID NO: 5 and a protein comprising at least 82% (e.g. at least 83%, 84%, 85%, 86%, 87%, 88%, 89% 90%, 91%, 92%, 93%, 94%, 95%, 96%, 97%, 98%, 99% or 100%) sequence identity to SEQ ID NO: 6.

Another aspect of the invention is an antibody raised against a protein or protein part selected from the group consisting of: a protein comprising at least 93% (e.g. at least 94%, 95%, 96%, 97%, 98%, 99% or 100%) sequence identity to SEQ ID NO: 3, a protein comprising at least 94% (e.g. at least 95%, 96%, 97%, 98%, 99% or 100%) sequence identity to SEQ ID NO: 4, a protein, comprising at least 80% (e.g. at least 81%, 82%, 83%, 84%, 85%, 86%, 87%, 88%, 89% 90%, 91%, 92%, 93%, 94%, 95%, 96%, 97%, 98%, 99% or 100%) sequence identity to SEQ ID NO: 5 and a protein comprising at least 82% (e.g. at least 83%, 84%, 85%, 86%, 87%, 88%, 89% 90%, 91%, 92%, 93%, 94%, 95%, 96%, 97%, 98%, 99% or 100%) sequence identity to SEQ ID NO: 6. Such an antibody can be used to detect ToMSV strains in plant tissues or in extracts of plant tissues. A kit comprising such an antibody is a further aspect of the invention. Such a kit may e.g. be an ELISA kit or a LFD kit.

Also use of the virus of the invention for identifying resistant plants or partially resistant plants of the genus Solanum or Capsicum is provided herein.

Likewise use of the nucleic acid molecules (or sequences), protein molecules (or sequences), or parts of any of these, for detection of the virus of the invention in a plant, plant part or in a sample is provided.

### DEPOSIT INFORMATION

A representative sample of the ToMSV strain Ve484 was deposited by Nunhems B.V. at the DSMZ (Deutsche Sammlung von Mikroorganismen and Zellkulturen GmbH, Inhoffenstrasse 7 B, 38124 Braunschweig, Germany) on Jan. 19, 2015, under accession number DSM 29970.

The Applicant requests that samples of the biological material and any material derived therefrom be only released to a designated Expert in accordance with Rule 32(1) EPC or related legislation of countries or treaties having similar rules and regulation, until the mention of the grant of the patent, or for 20 years from the date of filing if the application is refused, withdrawn or deemed to be withdrawn.

Access to the deposit will be available during the pendency of this application to persons determined by the Director of the U.S. Patent Office to be entitled thereto upon request. Subject to 37 C.F.R. § 1.808(b), all restrictions imposed by the depositor on the availability to the public of the deposited material will be irrevocably removed upon the granting of the patent. The deposit will be maintained for a period of 30 years, or 5 years after the most recent request, or for the enforceable life of the patent whichever is longer, and will be replaced if it ever becomes nonviable during that period. Applicant does not waive any rights granted under this patent on this application or under the Plant Variety Protection Act (7 USC 2321 et seq.).

The following non-limiting examples illustrate the production of pepper plants, seeds and fruits according to the invention. All references mentioned herein are incorporated by reference.

### EXAMPLES

### Example 1—Identification of ToMSV

Tomato plants with mosaic symptoms were identified in a leaf sample from a tomato production field. The tomato plants carried the Tm2² resistance gene in homozygous form.

### 1.1 Lateral Flow Device Test

Symptomatic leaves were collected and a Lateral Flow Device Test was carried out using an ImmunoStrip® test (ISK 57400/0025) for TMV (Tobacco Mosaic Virus) detected provided by Agdia, Inc. using the manufacturer's instructions. In short: Symptomatic leaf tissue is placed in an extraction bag (between mesh lining) containing SEB1 buffer. The virus is extracted by rubbing the bag between the mesh lining. Then the immunostrip is placed into the so-called channel portion of the bag for 30 minutes. A control line will become visible if the test was performed adequately. If virus is present also a pink/purple test line will appear on the strip. The antibody in the test detects a number of viruses (but not all) from the tobamovirus group.

The test was positive, indicating that the mosaic symptoms are caused by a member of the tobamovirus group.

### 1.2 Detection of the Virus in Tomato Seeds

Seeds were collected from infected plants found in the production field.

Two assays were carried out using the seeds, an ELISA assay (Agdia, Inc.) followed by a local lesion assay on Nicotiana tabacum plants as described in the ISF protocol “Method for the detection of infectious tobamoviruses on tomato seed” (found on the world wide web at worldseed.org/cms/medias/file/TradeIssues/PhytosanitaryMatters/SeedHealthTesting/ISHI-Veg/Tomato_Tobamo_Sept_2013.pdf).

### 1.2.1 Sample Preparation

Twelve inoculum samples were prepared from 250 tomato seeds each, by grinding the seed samples in extraction buffer. Also seeds from positive and negative controls samples were made. The inoculum samples were used first in the ELISA and then in the local lesion assay.

### 1.2.1 ELISA

The ELISA assay was carried out using the Tobacco Mosaic Virus ELISA Complete Kit (PSA 57400/0288) of Agdia, Inc. according to manufacturer's instructions. In short, this test uses a 96-well microtitre plate coated with an antibody that detects a number of viruses (but not all) from the tobamovirus group. Tissue samples (e.g. leaf tissue, seeds, etc.) are ground in extraction buffer and diluted. The samples are then loaded into the microtriter wells, together with the provided controls (positive control, negative control and buffer only) and incubated. After incubation the plates are washed and freshly prepared alkaline phosphatase enzyme conjugate is dispensed into the wells, which are then incubated again. The plates are washed again and PNP substrate is added to each well, which are then incubated again. The results are examined by eye and/or using a plate reader at 405 nm. Colored wells are positive for virus when examined by eye and/or a 405 nm reading of 2.5 times the background (negative control) is positive for the virus. Test results are only valid if the positive control is colored and the negative control is virtually clear and buffer-only wells are colorless.

### 1.2.2 the Local Lesion Assay

The local lesion assay is a bioassay which is used to detect infectious virus particles by mechanically inoculating leaves of Nicotiana tabacum var. Xanthi and Nicotiana glutinosa, both carrying the N gene for resistance against TMV. 3000 tomato seeds were used in the assay.

The Nicotiana plants were grown to the 4-5 true leaf stage and two leaves per plant were dusted with carborundum powder and inoculated with one of the 12 inoculum samples or with a control inoculum sample. Inoculation was done by rubbing a sponge with inoculum on the carborundum dusted leaf and smearing the entire surface of the leaf with the sponge. After inoculation the plants were incubated for 5-7 days at 20-25 degrees Celsius with at least 12 hours light and then the number of necrotic lesions on the inoculated leaves was recorded.

Such local necrotic lesions (hypersensitive response) show that one or more seeds of the 250 seed sample contained infectious virus particles.

### Results

The Tomato Mosaic Severe Virus strain was detected in tomato seeds of infected plants both in the ELISA assay and in the local lesion assay. The strain is therefore seed borne.

### 1.3 Sequencing and Sequence Analysis

Seed-extract inoculated tobacco leaves were used to prepare inoculum for mechanical inoculation of N. benthamiana leaves. The leaves were mechanically inoculated (using carborundum as described above) and the presence of virus particles in the inoculated leaves were confirmed by Electron Microscopy. RNA was isolated using the following primer combinations for RT-PCR (reverse transcriptase PCR) and sequencing:

Tob-Uni 1
(SEQ ID NO: 7)
5′-ATTTAAgTggASggAAAAVCACT-3′
Tob-Uni 2
(SEQ ID NO: 8)
5′-GTYGTTGATGAGTTCRTGGA-3′
or
Tobamo F
(SEQ ID NO: 9)
5′-GWCGCSGAKTCKGATTCGTWTTAAATATG-3′
Tobamo R
(SEQ ID NO: 10)
5′-TGGGCCSCTACCSGSGG-3′

Several different samples were sequenced and sequences were compared.

The genomic virus sequence is provided in SEQ ID NO: 1 and SEQ ID NO: 2 and in FIG. 1.

In SEQ ID NO: 1 ORF1 and ORF3 are shown, while in SEQ ID NO: 2 ORF2 (only final 492 amino acids are shown) and ORF4 are shown.

BLAST (Basic Local Alignment Search Tool) was carried out on the NCBI website using default parameters and also pairwise alignments using the program Needle were done with the best BLAST hits (using EMBOSS—needles, default parameters).

The BLAST result and pairwise alignment of the genome sequence of SEQ ID NO: 1 showed that the most similar virus in the database has only 82% sequence identity to the present virus, indicating that the present virus is a new species of tobamovirus. The most similar virus is Genbank accession FR878069.1 (Tobacco Mosaic Virus Strain Ohio V, complete genome, genomic RNA). See the world wide web at ncbi.nlm.nih.gov/nuccore/FR878069.1. The virus is also different from the recently sequenced ToMMV (Tomato Mottle Mosaic Virus) found in Mexico, USA and China (Genbank accession KF477193) with which it shares only 80.8% sequence identity.

Four ORFs were present in the genome. ORF1 encodes protein p126, provided in SEQ ID NO: 3. ORF2 encodes protein p183, provided in SEQ ID NO: 4. The protein of SEQ ID NO: 4 results from suppression of the stop codon at the end of ORF1. ORF3 encodes the movement protein, provided in SEQ ID NO: 5. ORF4 encodes the Coat Protein, provided in SEQ ID NO: 6.

The new species of tobamovirus is named herein Tomato Mosaic Severe Virus (ToMSV or TMSV) and the infectious strain sequenced and deposited is named VE484.

### Example 2—Bioassays of the New ToMSV Virus on Tomato and Pepper (Results not Shown)

Differential genotypes of tomato varieties comprising different TMV/ToMV resistance genes were inoculated mechanically with the deposited virus strain, VE484. The genotypes were inoculated in the biological assay were:

| Tomato variety | Genotype |
|---|---|
| Mobaci | Tm1/Tm1 (homozygous for Tm1) |
| Moperou | Tm2/Tm2 (homozygous for Tm2) |
| Momor | Tm2²/Tm2² (homozygous for Tm2²) |
| Mocimor | Tm2²Tm1/Tm2²Tm1 (homozygous for Tm1 and |
| | for Tm2²) |
| Philippos | Tm2²/ Tm2² (homozygous for Tm2²) |

All the plants were inoculated at 15 days stage and evaluated visually at 15 days post inoculation, when systemic symptoms were recorded. 12 plants per genotypes were inoculated.

These different genotypes showed a range of systemic symptoms: leaf mosaic, leaf distortion, blistering and bronzing. A LFD test was used to confirm the presence of the virus in the symptomatic leaves.

All plants showed symptoms and were therefore susceptible to the virus. The virus can therefore cause systemic symptoms on plants homozygous for Tm1 and/or Tm2 or Tm2² resistance genes.

| | | | | No of | No of | | Lateral |
|---|---|---|---|---|---|---|---|
| | | | No of plants with | plants | plants | No of plants | Flow |
| Tomato | | No. of | mosaic | with leaf | with leaf | with leaf | Device |
| variety | Genotype | plants | symptoms | distortion | blistering | bronzing | Test |
| Mobaci | Tm1/Tm1 | 12 | 3 | 5 | 4 | 0 | positive |
| Moperou | Tm2/Tm2 | 12 | 3 | 4 | 1 | 0 | positive |
| Momor | Tm2²/Tm2² | 12 | 8 | 2 | 5 | 10 | positive |
| Mocimor | Tm2²Tm1/ | 12 | 4 | 3 | 3 | 0 | positive |
| | Tm2²Tm1 | | | | | | |
| Philippos | Tm2²/Tm2² | 12 | 11 | 1 | 3 | 0 | positive |

The results were further confirmed in an ELISA test, where as a negative control a healthy, non-infect tomato variety was included and also, as positive control, the susceptible variety Monalbo was included (lacking Tm resistance genes) (data not shown). The results of the ELISA also confirmed that the new ToMSV virus strain VE484 can overcome all the known tobamovirus resistance genes, Tm1 (also referred to as Tm-1), Tm2 (also referred to as Tm-2) and Tm2² (also referred to as Tm-22).

1-18. (canceled)

19. A method for producing one or more Solanum or Capsicum plants comprising resistance against a tobamovirus whose genome comprises at least 95% sequence identity to SEQ ID NO: 1, comprising the steps of:
inoculating one or more plant parts of the genus Solanum or Capsicum with an inoculum comprising an infectious dose of the tobamovirus;
incubating the inoculated plants; and
producing one or more plants comprising resistance to the tobamovirus, wherein the virus causes systemic symptoms on Solanum lycopersicum plants homozygous for one or more Tm resistance genes of Tm1, Tm2, and/or Tm2².

20. The method of claim 19, further wherein the producing step comprises identifying one or more plants that do not develop systemic symptoms or plants that have reduced systemic symptoms after inoculation with the tobamovirus when compared to a susceptible control plant.

21. The method according to claim 20, wherein identifying further comprises assessing symptoms on the plants or plant parts.

22. The method according to claim 20, wherein identifying further comprises determining the presence of virus particles in non-inoculated plant parts.

23. The method of claim 19, wherein a plant comprising resistance to the tobamovirus has no systemic symptoms and in which the virus particles are not present in non-inoculated parts of the plant.

24. The method of claim 19, wherein a plant comprising resistance to the tobamovirus has local lesions on the inoculated plant part and/or in which the virus particles are not present in non-inoculated parts of the plant.

25. The method of claim 19, wherein a plant comprising resistance to the tobamovirus has no systemic symptoms and in which the virus particles are present in non-inoculated parts of the plant.

26. The method according to claim 19, wherein the plant part is a leaf, a cotyledon, a hypocotyl, a stem, a petiole or a root.

27. The method according to claim 19, wherein the Solanum or Capsicum plant is a cultivated tomato plant, a cultivated pepper plant, a cultivated aubergine plant or a cultivated pepino plant.

28. The method according to claim 19, wherein the plant of the genus Solanum is a Solanum lycopersicum, Solanum melongena, Solanum muricatum, Solanum arcanum, Solanum chmielewskii, Solanum neorickii, Solanum cheesmaniae, Solanum galapagense, Solanum pimpinellifolium, Solanum chilense, Solanum corneliomulleri, Solanum habrochaites, Solanum huaylasense, Solanum sisymbriifolium, Solanum peruvianum, Solanum hirsutum, Solanum pennellii, Solanum lycopersicoides, Solanum sitiens, or Solanum ochranthum plant.

29. The method of claim 19, further comprising crossing the plant comprising resistance to the virus with a susceptible plant to produce an F₁ generation; and selecting one or more plants comprising the resistance against the tobamovirus from the F₁ generation.

30. The method according to claim 20, wherein identifying further comprises assessing symptoms on the plants or plant parts and determining the presence of virus particles in non-inoculated plant parts.

31. The method according to claim 30, wherein the presence of virus particles is determined using microscopy, an antibody based assay, or a test that detects RNA or cDNA.

32. The method according to claim 31, wherein the microscopy is electron microscopy.

33. The method according to claim 31, wherein the antibody based test is an ELISA or lateral flow device test.

34. The method according to claim 31, wherein the test that detects virus RNA or cDNA is a Polymerase Chain Reaction (PCR) based method, a nucleic acid hybridization method, or a bioassay.

35. The method of claim 19, wherein the virus causes systemic symptoms on Solanum lycopersicum plants homozygous for Tm2².

36. The method of claim 19, wherein the virus causes systemic symptoms on Solanum lycopersicum plants homozygous for Tm1, Tm2, and Tm2².

37. The method of claim 19, wherein the method produces a cultivated plant comprising resistance against a tobamovirus whose genome comprises at least 97% sequence identity to SEQ ID NO: 1.

38. The method of claim 19, wherein the method produces a cultivated plant comprising resistance against a tobamovirus whose genome comprises at least 99% sequence identity to SEQ ID NO: 1.

39. The method of claim 35, wherein the method produces a cultivated plant comprising resistance against a tobamovirus whose genome comprises at least 97% sequence identity to SEQ ID NO: 1.

40. The method of claim 35, wherein the method produces a cultivated plant comprising resistance against a tobamovirus whose genome comprises at least 99% sequence identity to SEQ ID NO: 1.

41. The method of claim 36, wherein the method produces a cultivated plant comprising resistance against a tobamovirus whose genome comprises at least 97% sequence identity to SEQ ID NO: 1.

42. The method of claim 36, wherein the method produces a cultivated plant comprising resistance against a tobamovirus whose genome comprises at least 99% sequence identity to SEQ ID NO: 1.


SEQUENCE LISTING (11 sequences)

SEQ ID NO: 1 - DNA, 6402 bp, unidentified
  source 1..6402: mol_type = genomic DNA
  exon 77..3424: note = Protein sequence of the exon is shown by protein Seq-ID 3; note = ORF1 = p126
  exon 4911..5708: note = ORF3 = Movement protein; note = Protein sequence of the exon is shown by protein Seq-ID 5
  misc_feature 1..6402: note = genome of new tobamovirus
gtatttttgttttacaacatataccaacaacaacaaacaacaaacaacaacattacaattactatttacaactacaatggcatacacacagacagctaccacatccgctttgctcgacactgtccgaggtaacaataccttggtcaatgatcttgcgaagcggcgtctttatgacacagcggtcgacgagttcaacgctcgtgatcgcaggcccaaagtaaatttttccaaagtaataagtgaggaacagacgcttattgctactagggcatatccagaattccagataaccttctataatacgcagaacgccgtgcattcgcttgccggtggactacgatccttagaactggaatatctaatgatgcagatcccgtacggatcactcacatatgatataggtgggaattttgcatctcatctgttcaaaggacgggcatatgttcactgctgtatgcccaatcttgatgtccgcgacataatgcggcacgaaggccagaaagacagtatagaattatacctttccaggcttgagcggggcaacaaagttgtcccaaatttccaaaaggaagctcttgacagatacgctgaaacgccagacgaagttgtctgtcacagtaccttccaaacgtgtacgcaccagcaggtggaaaacacaggcagggtgtatgctattgcattgcacagtatatacgatatacctgctgatgaattcggagcggcacttttaaggaaaaatgtccatgtttgttacgccgccttccacttttccgagaatttacttctcgaagattcacacgtcaaccttgacgaaatcaacgcgtgtttttcgcgtgatggagacaagctgactttttctttcgcatctgagagcactttaaattattgtcatagttattctaatattttaaaatacgtgtgcaaaacttacttcccggcatctaatagagaggtctacatgaaggagtttttggtcaccagggttaacacctggttttgtaagttttctaggatagatacttttttattatacaagggggtagcccacaaaggtgtaaatagtgagcaattttacagcgcaatggaagatgcatggcactacaaaaagactcttgcaatgtgtaacagcgagaggattcttcttgaagattcctcatcggtcaattactggttcccaaaaatgagagatatggtcatagttcctctattcgacatatctctcgacaccagtaaaaggacccgcaaagaagtcttagtgtcaaaggattttgtattcacagttttaaatcacattcgcacttatcaagccaaggcacttacatactccaatgttttatcctttgtcgaatcaattcgttcaagggtaattatcaacggagtgactgccaggtctgagtgggatgttgacaaatctcttttgcaatccttgtccatgacatttttcttgcatactaagcttgccgttttaaaagacgaattgttaatcagcaagtttagtttggggccaaaatcagtaagccagcatgtatgggatgagatttccctggcttttggaaacgcatttccatcgatcaaggagagactgctaaatcggaaactaattaaagtgtcgggagacgcattagaaatcagggtgcctgatttatatgtgacttttcacgatagattagtgactgagtacaaaacatcggtggatatgccagtgcttgatatcagaaagagaatggaggagactgaggttatgtacaatgcattgtctgagctatctgtgctcaaggagtcggacaagttcgacgctgatgttttttcccggatgtgccagactttggaggtagacccaatgactgcagcaaaggttattgtggcagtgatgagcaacgagagcggactgactcttacattcgaacagccaactgaagcaaatgtcgcattggcacttaaagattcagaaaaagcctctgagggtgcactagtggttacttctagagatgttgaagaaccatccatgaagggttcaatggcaagaggagagttacaattggccggtctgtctggagaccaaccagagtcttcctatactcggaacgaggaaatagagtcattagagcaattccacatggcaacggctagttcgttaattcggaaacagatgagttcgattgtgtacacgggccccattaaagttcagcaaatgaaaaactttattgatagcctggtagcatcactctctgctgcggtgtcgaacctagtcaagatcctaaaggatacagctgctatagatctcgaaacccgtcagaagtttggagtcttagatgttgcgaccaaaagatggttaattaaacctttagccaagaatcacgcatggggcgttattgaaacacatgctaggaagtaccacgttgcacttttggagtatgatgagcatggagtggtaacttgcgacagttggagaagggtggccgtgagttctgagtcaatggtttattctgatatggcgaagctcagaacactgaggagattattaagagatggtgagcctcatgtcagcagtgctaaagtcgtcctagttgacggtgtcccgggttgtggaaagacaaaagagattctctcgaaagtaaattttgaggaagatctaatcttagtaccgggtaagcaggctgctgaaatgataaagaggcgtgctaatgcgtcaggaataattcaagccacaagagataatgttcgtactgttgattcatttataatgaattacggtaaaggaacacgctgtcagttcaaaaggttatttatcgacgaaggtctgatgttgcacactggttgtgtgaattttcttgtttctatgtctctgtgcgaaattgcatatgtttatggagacacacaacaaattccatacatcaacagagtatccggttttccgtaccctgcacattttgcaaaaatagaggttgatgaggtggaaactcgcagaactacgctgcgttgtccagccgacattacccactatcttaacagaaggtacgaaggatatgtcatgtgtacatcgtcggttaaaaagtcagtttctcaggaaatggtgagcggggccgcaatgatcaatcctgtatctaagccattgaatgggaaagttttgactttcactcagtctgataaagaggcgctgctttctcgaggatatacggacgtccatacagtacatgaggtacaaggtgagacatatgcagatgtgtcgttggtcagattgactccgacacctgtatctatcatcgcaggagatagtccgcacgttctcgtagctttgtcaaggcatacccaaacattgaagtattacaccgtagtgatggatcctcttgtaagtataattagggatttagaaaaacttagttcttacttgttagatatgtataaagtagatgcagggacccaatagcaattacaggtagactccgtgtttaaaggttctaatctttttgttgcagcaccaaagactggagatatctcagatatgcaattttactatgataagtgtctcccaggtaatagcaccatgttaaataactatgatgctgttaccatgaggttgactgacatttctcttaatgtcaaagattgcatattggatttctctaagtctgtggctgcaccgaaggatccgatcaaaccactgattccaatggtacgaacagcggcagaaatgccacgccagactggactattggaaaatttggtggcgatgatcaaaagaaactttaattcaccggagttatcaggaataatcgacattgagaatactgcatctttagtagtagataaattttttgatagttacttgcttaaagaaaaaagaaaaccaaataaaaatgtttctttattttgtagagagtctctcaatagatggttagagaagcaggagcaagtgaccattggtcagcttgcagattttgattttgtggatcttcctgccgttgatcagtacaggcatatgattaaagcgcaacctaagcagaagctggatacatcaattcaaagcgaatatccggccttgcagacgattgtgtatcattcgaaaaagatcaacgcaatcttcggtcctttgttcagtgagctcacaaggcaaatgctcgaaagcatagactcaagtaagtttttgttctttacaaggaagacgccagctcaaattgaggatttcttcggagatctcgatagccatgtccctatggatatcttggagttggatatttcgaagtatgacaaatctcagaacgagttccactgtgcagtagagtatgaaatatggagaagacttggattagaagattttctgggagaagtttggaaacaaggccataggaaaactactcttaaagattacacagctggtattaaaacgtgtttatggtaccagagaaagagtggggacgttacaacattcatcggtaatacggtgattattgctgcttgtttagcttccatgttgcccatggagaaaataatcaaaggtgcattttgcggagatgacagtttactatacttcccaaaaggttgtgagtttcctgacatacagcatacagccaaccttatgtggaatttcgaggctaagctattcagaaagcagtatggttatttctgtggaaggtacgtgatacatcatgacagagggtgtattgtttattatgaccctttgaagttgatttctaaacttggtgctaaacacatcaaggattgggatcacttagaagagttcagaagatccctttgtgatgttgcaaattcgttgaacaactgtgcgtattacacgcagttggacgacgctgtgagtgaggtccataaaaccgcacccccgggttcgtctgtatataaaagtttagttaaatatctgtccgataaggttctttttagaagtttgtttatagatggctcttgttaagggtaaagtcaatattaatgagttcatagacttgtcaaaatcagaaaaatttcttccgtctatgttcacacctgttaagagtgtcatgatctccaaggttgataagatattggttcatgaagatgaatctttgtccgaagtcaatttactcaaaggtgtaaaactcattgatggtggctatgtacatcttgctggtcttgtggtgacaggtgaatggaatttgccagataattgtcgtggtggtgtcagtgtctgtttggtcgataagagaatggagagagcggacgaggcaactcttgcttcatactataccgcagcggctaagaaaaggtttcagttcaaagtcgttccaaattacaacatcactaccaaggacgcagaaaaggcagtttggcaagtactagttaatattagaaatgttaaaattgctgcgggttactgtccgctgtcattagaatttgtgtcagtgtgtattgtttataaaaatattataaaactcggtttgagagagaaaattacgagcgtcacggatggagggcccatggaactatcagaagaagttgttgatgagttcatggaagaagtcccgatgtctgtaaggcttgcaaaatttcgttcgaagaccggaaaaaagtttagtagtaaaagtgagaataatagtggtaataataggccgaaaccagacaaaaaccaaaggaagggaaagggtttaaaagttagggttgagaaggataatttaattgataatgaattggagacttacgtcgccgattcagattcgtattaaatatttaaatatgtcttacacaatcgcaactccatcgcaatttgtgtttttgtcatcagcatgggccgaccctatagaattaataaatttatgtactaattcactaggtaatcagttccaaacacaacaagctagaacaaccgttcaacggcaatttagcgaagtgtggaaacctgtccctcaagtcactgttaggtttcctgacagtggttttaaggtgtataggtacaatgcggtactagatcctctagttactgctttgttaggagctttcgatactagaaataggattatagaagtcgaaaatcaggcgaacccgacaaccgccgaaacgttagacgctactcgtagagtagatgacgcaacggtggctataaggagcgctataaataatttagtagtagaattggtcaaaggaacaggtttgtacaatcagagcacatttgaaagtgcatccggtttacaatggtcctctgcacctgcatcttgagataatcgagatgcttaaataacagattgtgtctgcaaacacacgtggtacgtacgataacgtatagtgtttttccctccacttaaatcgaagggtagtgtcttggagcgcgcgggacaaatgtgtatggttcatacacatccgtaggcacgtaataaagcgagggattcgaattcccccggaacccccggtaggggccca

SEQ ID NO: 2 - DNA, 6402 bp, unidentified; identical to SEQ ID NO: 1
  source 1..6402: mol_type = genomic DNA
  exon 3446..4921: note = ORF2 (p183) final 492 amino acids; note = Protein sequence of the exon is shown by protein Seq-ID 11
  exon 5671..6198: note = ORF4 = Coat Protein; note = Protein sequence of the exon is shown by protein Seq-ID 6
  misc_feature 1..6402: note = genome of new tobamovirus

SEQ ID NO: 3 - protein, 1116 aa, unidentified
  source 1..1116: note = ORF 1 (protein p126) of ToPV strain VE484
  REGION 1..1116: note = ORF 1 (protein p126) of ToPV strain VE484
MAYTQTATTSALLDTVRGNNTLVNDLAKRRLYDTAVDEFNARDRRPKVNFSKVISEEQTLIATRAYPEFQITFYNTQNAVHSLAGGLRSLELEYLMMQIPYGSLTYDIGGNFASHLFKGRAYVHCCMPNLDVRDIMRHEGQKDSIELYLSRLERGNKVVPNFQKEALDRYAETPDEVVCHSTFQTCTHQQVENTGRVYAIALHSIYDIPADEFGAALLRKNVHVCYAAFHFSENLLLEDSHVNLDEINACFSRDGDKLTFSFASESTLNYCHSYSNILKYVCKTYFPASNREVYMKEFLVTRVNTWFCKFSRIDTFLLYKGVAHKGVNSEQFYSAMEDAWHYKKTLAMCNSERILLEDSSSVNYWFPKMRDMVIVPLFDISLDTSKRTRKEVLVSKDFVFTVLNHIRTYQAKALTYSNVLSFVESIRSRVIINGVTARSEWDVDKSLLQSLSMTFFLHTKLAVLKDELLISKFSLGPKSVSQHVWDEISLAFGNAFPSIKERLLNRKLIKVSGDALEIRVPDLYVTFHDRLVTEYKTSVDMPVLDIRKRMEETEVMYNALSELSVLKESDKFDADVFSRMCQTLEVDPMTAAKVIVAVMSNESGLTLTFEQPTEANVALALKDSEKASEGALVVTSRDVEEPSMKGSMARGELQLAGLSGDQPESSYTRNEEIESLEQFHMATASSLIRKQMSSIVYTGPIKVQQMKNFIDSLVASLSAAVSNLVKILKDTAAIDLETRQKFGVLDVATKRWLIKPLAKNHAWGVIETHARKYHVALLEYDEHGVVTCDSWRRVAVSSESMVYSDMAKLRTLRRLLRDGEPHVSSAKVVLVDGVPGCGKTKEILSKVNFEEDLILVPGKQAAEMIKRRANASGIIQATRDNVRTVDSFIMNYGKGTRCQFKRLFIDEGLMLHTGCVNFLVSMSLCEIAYVYGDTQQIPYINRVSGFPYPAHFAKIEVDEVETRRTTLRCPADITHYLNRRYEGYVMCTSSVKKSVSQEMVSGAAMINPVSKPLNGKVLTFTQSDKEALLSRGYTDVHTVHEVQGETYADVSLVRLTPTPVSIIAGDSPHVLVALSRHTQTLKYYTVVMDPLVSIIRDLEKLSSYLLDMYKVDAGTQ

SEQ ID NO: 4 - protein, 1609 aa, unidentified
  REGION 1..1609: note = ToPV strain VE484 - ORF2 - protein p183
  VARIANT 1117: note = X (Xaa) can be any naturally occurring amino acid
MAYTQTATTSALLDTVRGNNTLVNDLAKRRLYDTAVDEFNARDRRPKVNFSKVISEEQTLIATRAYPEFQITFYNTQNAVHSLAGGLRSLELEYLMMQIPYGSLTYDIGGNFASHLFKGRAYVHCCMPNLDVRDIMRHEGQKDSIELYLSRLERGNKVVPNFQKEALDRYAETPDEVVCHSTFQTCTHQQVENTGRVYAIALHSIYDIPADEFGAALLRKNVHVCYAAFHFSENLLLEDSHVNLDEINACFSRDGDKLTFSFASESTLNYCHSYSNILKYVCKTYFPASNREVYMKEFLVTRVNTWFCKFSRIDTFLLYKGVAHKGVNSEQFYSAMEDAWHYKKTLAMCNSERILLEDSSSVNYWFPKMRDMVIVPLFDISLDTSKRTRKEVLVSKDFVFTVLNHIRTYQAKALTYSNVLSFVESIRSRVIINGVTARSEWDVDKSLLQSLSMTFFLHTKLAVLKDELLISKFSLGPKSVSQHVWDEISLAFGNAFPSIKERLLNRKLIKVSGDALEIRVPDLYVTFHDRLVTEYKTSVDMPVLDIRKRMEETEVMYNALSELSVLKESDKFDADVFSRMCQTLEVDPMTAAKVIVAVMSNESGLTLTFEQPTEANVALALKDSEKASEGALVVTSRDVEEPSMKGSMARGELQLAGLSGDQPESSYTRNEEIESLEQFHMATASSLIRKQMSSIVYTGPIKVQQMKNFIDSLVASLSAAVSNLVKILKDTAAIDLETRQKFGVLDVATKRWLIKPLAKNHAWGVIETHARKYHVALLEYDEHGVVTCDSWRRVAVSSESMVYSDMAKLRTLRRLLRDGEPHVSSAKVVLVDGVPGCGKTKEILSKVNFEEDLILVPGKQAAEMIKRRANASGIIQATRDNVRTVDSFIMNYGKGTRCQFKRLFIDEGLMLHTGCVNFLVSMSLCEIAYVYGDTQQIPYINRVSGFPYPAHFAKIEVDEVETRRTTLRCPADITHYLNRRYEGYVMCTSSVKKSVSQEMVSGAAMINPVSKPLNGKVLTFTQSDKEALLSRGYTDVHTVHEVQGETYADVSLVRLTPTPVSIIAGDSPHVLVALSRHTQTLKYYTVVMDPLVSIIRDLEKLSSYLLDMYKVDAGTQXVFKGSNLFVAAPKTGDISDMQFYYDKCLPGNSTMLNNYDAVTMRLTDISLNVKDCILDFSKSVAAPKDPIKPLIPMVRTAAEMPRQTGLLENLVAMIKRNFNSPELSGIIDIENTASLVVDKFFDSYLLKEKRKPNKNVSLFCRESLNRWLEKQEQVTIGQLADFDFVDLPAVDQYRHMIKAQPKQKLDTSIQSEYPALQTIVYHSKKINAIFGPLFSELTRQMLESIDSSKFLFFTRKTPAQIEDFFGDLDSHVPMDILELDISKYDKSQNEFHCAVEYEIWRRLGLEDFLGEVWKQGHRKTTLKDYTAGIKTCLWYQRKSGDVTTFIGNTVIIAACLASMLPMEKIIKGAFCGDDSLLYFPKGCEFPDIQHTANLMWNFEAKLFRKQYGYFCGRYVIHHDRGCIVYYDPLKLISKLGAKHIKDWDHLEEFRRSLCDVANSLNNCAYYTQLDDAVSEVHKTAPPGSSVYKSLVKYLSDKVLFRSLFIDGSC

SEQ ID NO: 5 - protein, 266 aa, unidentified
  REGION 1..266: note = ToPV VE484 - ORF3 - Movement Protein
MALVKGKVNINEFIDLSKSEKFLPSMFTPVKSVMISKVDKILVHEDESLSEVNLLKGVKLIDGGYVHLAGLVVTGEWNLPDNCRGGVSVCLVDKRMERADEATLASYYTAAAKKRFQFKVVPNYNITTKDAEKAVWQVLVNIRNVKIAAGYCPLSLEFVSVCIVYKNIIKLGLREKITSVTDGGPMELSEEVVDEFMEEVPMSVRLAKFRSKTGKKFSSKSENNSGNNRPKPDKNQRKGKGLKVRVEKDNLIDNELETYVADSDSY

SEQ ID NO: 6 - protein, 176 aa, unidentified
  REGION 1..176: note = ORF4 - Coat Protein of ToPV VE484
MNWRLTSPIQIRIKYLNMSYTIATPSQFVFLSSAWADPIELINLCTNSLGNQFQTQQARTTVQRQFSEVWKPVPQVTVRFPDSGFKVYRYNAVLDPLVTALLGAFDTRNRIIEVENQANPTTAETLDATRRVDDATVAIRSAINNLVVELVKGTGLYNQSTFESASGLQWSSAPAS

SEQ ID NO: 7 - DNA, 23 bp, unidentified
  source 1..23: mol_type = unassigned DNA
  misc_feature 1..23: note = primer Tob-uni 1
atttaagtggasggaaaavcact

SEQ ID NO: 8 - DNA, 20 bp, unidentified
  source 1..20: mol_type = unassigned DNA
  misc_feature 1..20: note = primer Tob-uni 2
gtygttgatgagttcrtgga

SEQ ID NO: 9 - DNA, 29 bp, unidentified
  source 1..29: mol_type = unassigned DNA
  misc_feature 1..29: note = primer Tobamo F
gwcgcsgaktckgattcgtwttaaatatg

SEQ ID NO: 10 - DNA, 17 bp, unidentified
  source 1..17: mol_type = unassigned DNA
  misc_feature 1..17: note = primer tobamo R
tgggccsctaccsgsgg

SEQ ID NO: 11 - protein, 492 aa, unidentified
  REGION 1..492: note = ORF2 (p183) final 492 amino acids: protein sequence of exon pos 3446 to 4921 of Seq-ID 2
VFKGSNLFVAAPKTGDISDMQFYYDKCLPGNSTMLNNYDAVTMRLTDISLNVKDCILDFSKSVAAPKDPIKPLIPMVRTAAEMPRQTGLLENLVAMIKRNFNSPELSGIIDIENTASLVVDKFFDSYLLKEKRKPNKNVSLFCRESLNRWLEKQEQVTIGQLADFDFVDLPAVDQYRHMIKAQPKQKLDTSIQSEYPALQTIVYHSKKINAIFGPLFSELTRQMLESIDSSKFLFFTRKTPAQIEDFFGDLDSHVPMDILELDISKYDKSQNEFHCAVEYEIWRRLGLEDFLGEVWKQGHRKTTLKDYTAGIKTCLWYQRKSGDVTTFIGNTVIIAACLASMLPMEKIIKGAFCGDDSLLYFPKGCEFPDIQHTANLMWNFEAKLFRKQYGYFCGRYVIHHDRGCIVYYDPLKLISKLGAKHIKDWDHLEEFRRSLCDVANSLNNCAYYTQLDDAVSEVHKTAPPGSSVYKSLVKYLSDKVLFRSLFIDGSC